(19) United States
(12) Patent Application Publication
(10) Pub. No.: US 2007/0275420 A1
(43) Pub. Date: Nov. 29, 2007

(54) Method For Detecting Prognosis Of Cancer

(76) Inventors: Chikara Ohyama, Sendai-shi (JP); Minoru Fukuda, San Diego, CA (US)

Correspondence Address: SUGHRUE MION, PLLC, 2100 PENNSYLVANIA AVENUE, N.W., SUITE 800, WASHINGTON, DC, 20037, US

(21) Appl. No.: 10/549,811

(22) PCT Filed: Mar. 19, 2004
(86) PCT No.: PCT/US04/06085

§ 371 Date: Sep. 14, 2006

Related U.S. Application Data
(60) Provisional application No. 60/455,585, filed on Mar. 19, 2003.

(51) Int. Cl. G01N 33/574; C07K 16/40

(52) U.S. Cl. 435/7.23; 530/388.26

(57) ABSTRACT

It is to provide a method and a kit for detecting the prognosis of cancer at high accuracy in a simple and rapid manner at low cost. The method is specifically a method for detecting the prognosis of cancer, at least including a step of detecting core-2 β 1,6-acetylglucosaminyltransferase in a sample collected from a biological organism to examine the relationship between the results of the detection and the prognosis of cancer in the biological organism, wherein core-2 β1,6-acetylglucosaminyltransferase is preferably core-2 β1,6-acetylglucosaminyltransferase-I; the biological organism is preferably human body; and the sample is preferably a living tissue.

This application is based on and claims priority from U.S. Provisional Application No. 60/455,585, filed Mar. 19, 2003.

### TECHNICAL FIELD

The present invention relates to a method for detecting the prognosis of cancer and a kit used for the detection.

### BACKGROUND ART

First of all, the following abbreviations used in this specification are now described below.

C2GnT: core-2 β1,6-N-acetylglucosaminyltransferase

C2GnT-I: core-2 β1,6-N-acetylglucosaminyltransferase-I

The C2GnT-I referred to in this specification is the C2GnT specified as “GenBank accession No. M97347”.

GnT-V: N-acetylglucosaminyltransferase-V

PSA: prostate specific antigen

sLe^{x}: sialyl Lewis^{x}

C2GnT and GnT-V are glycosyltransferases responsible for the control of the branching structures of glycoprotein sugar chains. Clin. Cancer Res., 6, 1772-1777 (2000) discloses that GnT-V and its product branching N-glycan have a correlation with the metastatic potency of colorectal cancer. Further, Int. J Cancer, 91, 631-637 (2001) discloses that the expression of GnT-V is observed as an early event of carcinogenesis in case of liver cell carcinoma and the expression thereof is rather reduced in cases with metastasis. C2GnT is a branching enzyme of O-glycans to generate the core2 structure. It is known that like GnT-V, C2GnT generates β1,6-branched sugar chains and expression of C2GnT has a correlation with the metastatic potencies of various cancer cells. Cancer Res, 57, 5201-5206 (1997) and Cancer Res., 6, 2226-2231 (2001) disclose that the expression of C2GnT has high correlation with the depth of cancer invasion and the metastatic potency to lymph node.

However, no relationship between C2GnT and the prognosis of cancer has been known.

Prostate cancer as one cancer type is generally detected with PSA. Generally, total resection is applied to prostate cancer (prostatectomy). However, the surgical mode has been diversified lately. Specifically, the surgical mode includes total resection by laparotomy and lymph node resection, laparoscopic resection, and resection by perineotomy and optionally includes follow-up observations in case of a cancer at slow progression until the symptoms thereof develop.

The parameters used in the determination of such diverse therapeutic courses include for example Gleason score, PSA and the clinical TNM classification. However, it is difficult to accurately predict the prognosis of cancer post-treatment at the time when not any therapeutic treatment is yet conducted on the cancer, on the basis of these parameters alone. When the prognosis of cancer, for example prostate cancer after treatment can be accurately predicted on the basis of the data before the therapeutic treatment (before prostatectomy, for example) of the cancer, very useful information for the determination of a therapeutic course for the cancer can be provided, including for example the presence or absence of the need of for example prostatectomy, the possibility of radical cure of the cancer by for example prostatectomy alone, and the need of selection of another therapeutic treatment. This leads to the avoidance of any unnecessary medical treatment and to the provision of such information at high accuracy to such patients, advantageously for these patients's benefit.

### DISCLOSURE OF THE INVENTION

An object of the present invention is to provide a method for detecting the prognosis of cancer at high accuracy in a simple and rapid manner at low cost and a kit for the detection.

The inventors have made investigations so as to solve the problems. The inventors have found that the prognosis of cancer can be examined and predicted by detecting C2GnT. Based on this finding, the inventors have found a method and a kit for detecting the prognosis of cancer, which are now provided. Thus, the present invention has been achieved.

In other words, the present invention provides a method for detecting the prognosis of cancer (hereinafter referred to as the “inventive method”), at least including a step of detecting C2GnT in a sample collected from a biological organism to examine the relationship between the results of the detection and the prognosis of cancer in the biological organism. The “C2GnT” herein referred to is preferably “C2GnT-I”. Additionally, the biological organism herein referred to is preferably human body, while the sample is preferably a living tissue.

The detection of C2GnT is preferably done, using a polypeptide capable of binding to C2GnT. The “polypeptide” herein referred to is preferably an antibody or a polypeptide having its antigen-binding site.

The subject cancer of which the prognosis is examined and predicted is preferably one or two or more cancers selected from the group consisting of prostate cancer, testicular tumor and bladder cancer. The “prognosis of cancer” is preferably the “possibility of cancer metastasis or recurrence”.

The inventive method is preferably carried out before the resection or the dissection of cancer tissues. The “resection” is preferably total resection.

Additionally, the present invention provides a kit for detecting the prognosis of cancer, including at least the following element (A) (hereinafter referred to as “inventive kit”):

(A) a first polypeptide capable of binding to C2GnT.

Additionally, the inventive kit preferably includes at least the following element (B):

(B) a second polypeptide capable of specifically binding to the first polypeptide (A), the second polypeptide being labeled or capable of being labeled with a labeling substance.

The “polypeptide” is preferably an antibody or a polypeptide having its antigen-binding site.

### BRIEF DESCRIPTION OF THE DRAWINGS

FIG. 1 shows one immunostaining example of prostate cancer tissue with the anti-C2GnT antibody.

FIG. 2 shows the relationship between the time period after prostatectomy and PSA failure (PSA non-recurrence ratio).

FIG. 3 shows one immunostaining example of testis tumor tissue with the anti-C2GnT antibody.

FIG. 4 shows one immunostaining example of testicular tumor tissues at various malignancy levels with the anti-C2GnT antibody.

FIG. 5 shows one immunostaining example of testicular tumor tissues at various malignancy levels with the anti-GnT-V antibody.

FIG. 6 shows one immunostaining example of bladder cancer tissue with the anti-C2GnT antibody or with the anti-GnT-V antibody.

### BEST MODE FOR CARRYING OUT THE INVENTION

The embodiments for carrying out the present invention are described below.

<1> Inventive Method

The inventive method is a method for detecting the prognosis of cancer, at least including a step of detecting C2GnT in a sample collected from a biological organism to examine the relationship between the results of the detection and the prognosis of cancer in the biological organism.

Herein, the C2GnT is preferably C2GnT-I (the C2GnT specified as GenBank accession No. M97347).

The biological organism as a subject for sample collection is preferably a vertebrate animal, more preferably a mammalian animal. Specifically, human body is particularly preferable.

In accordance with the inventive method, the sample includes but is not limited to any sample from biological organisms, for example living tissues and body fluids [for example, urine, blood (under the concept that the term blood includes serum and plasma in this specification), saliva, sweat, tear fluid, joint fluid, extracts of cartilage, cell culture supernatants, etc.] and the like. Among these, living tissues are preferable. More preferably, the sample is derived from a living tissue with cancer, of which the prognosis is desirably examined and predicted.

Depending on the type of a sample, a person skilled in the art can appropriately select a method for collecting the sample from a biological organism. For use as such sample, a living tissue can be collected for example by routine biopsy. When a collected sample is “a living tissue”, a section specimen is preferably prepared from the living tissue. The method for preparing such section specimen includes but is not limited to general methods. For example, such section specimen can be prepared by fixing a collected living tissue in formalin, embedding the living tissue in paraffin, and thinly slicing the embedded tissue.

The method for detecting C2GnT in a sample includes but is not limited to any method possibly detecting C2GnT in some specific manner, for example a detection method using a substance binds to C2GnT, and a detection method including C2GnT extraction from a sample to detect C2GnT based on the physico-chemical properties and enzymatic properties thereof. Specifically, C2GnT detection is carried out by a method using a polypeptide capable of binding to C2GnT. More preferably, the detection is carried out by a method using a polypeptide capable of specifically binding to C2GnT.

Preferably, such “polypeptide” is an antibody or a polypeptide having its antigen-binding site (Fab). The “polypeptide having the Fab of an antibody” includes for example fragments containing the Fab of an antibody. The fragments containing such Fab can be prepared by treating an antibody with a protease never decomposing Fab (for example plasmin, pepsin, papain, etc.). The “fragment containing Fab” includes for example Fabc and (Fab′)₂ other than Fab.

Additionally, the “polypeptide having the Fab of an antibody” includes for example a chimera antibody with the intended Fab. Once the nucleotide sequence of the gene encoding an antibody or the amino acid sequence of an antibody is determined, a chimera antibody with the intended Fab region and a fragment containing the intended Fab region can be produced by genetic engineering.

Preferably, the “polypeptide” is preliminarily purified. When the polypeptide is “an antibody” and the immunoglobulin class thereof is IgG, for example, the polypeptide can be purified by affinity chromatography with protein A or protein G. When the immunoglobulin class of the antibody is IgM, the polypeptide can be purified by gel filtration column chromatography.

The “antibody” used herein is not specifically limited as long as the antibody can specifically bind to C2GnT. The antibody may be either polyclonal or monoclonal, satisfactorily. From the standpoints of specificity, homogeneity, reproducibility, and large-scale and consistent productivity, preferably, the antibody is a monoclonal antibody. Such antibody can be prepared by known methods.

When C2GnT is detected with the “polypeptide capable of binding to C2GnT”, a sample is put in contact to the polypeptide, to detect the polypeptide bound to the C2GnT in the sample. The contact of the “sample” to the “polypeptide” is done by any method with no limitation, as long as the method can put the C2GnT molecule in the sample at a state in contact to the polypeptide molecule.

In order to sufficiently bind the C2GnT molecule in the sample to the polypeptide molecule after these two are put in contact together, preferably, the resulting mixture reacts together, for example, at 4 to 37° C., preferably at about 20° C., for about one hour.

After the reaction, the solid phase and the liquid phase are separated from each other. If necessary, the surface of the solid phase is preferably rinsed with a rinse solution to discard the nonspecifically adsorbed matters and the polypeptide unreactive.

As the rinse solution, preferably, buffers [for example, phosphate buffer, phosphate-buffered physiological saline (PBS), Tris-HCl buffer, etc.] with addition of nonionic surfactants such as Tween-based surfactants are used.

The polypeptide is preferably labeled or can be labeled with a labeling substance, for easier detection. The labeling substance for use in the labeling includes but is not limited to any general labeling substances for use in protein labeling, for example enzymes (peroxidase, alkaliphosphatase, β-galactosidase, luciferase, acetylcholinesterase, glucose oxidase, etc.), radioactive elements (¹²⁵I, ¹³¹I, ³H, etc.), fluorescent dyes [fluorescein isothiocyanate (FITC), 7-amino-4-methylcoumarine-3-acetic acid (AMCA), dichlorotriazinylaminofluorescein (DTAF), tetramethylrhodamine isothiocyanate (TRITC), lissamine rhodamine B, Texas Red, phycoerythrin (PE), umbelliferone, europium, phycocyanine, tricolor, cyanine, etc.], chemiluminescent substances (luminol, etc.), haptens [dinitrofluorobenzene, adenosine monophosphate (AMP), 2,4-dinitroaniline, etc.], either one substance in one of specific binding pairs {biotin and avidins (streptoavidin, etc.), lectin and sugars, agonists and agonist receptors, heparin and anti-thrombin III (ATIII), polysaccharides and their binding proteins [hyaluronic acid and hyaluronic acid-binding protein (HABP)]}.

Even when the “polypeptide capable of binding to C2GnT” of itself is not labeled, additionally, a polypeptide (second polypeptide) specifically binding to the polypeptide (first polypeptide) may satisfactorily be used to detect the first polypeptide. The “second polypeptide” is not specifically limited as long as the second polypeptide specifically binds to the first polypeptide. When the first polypeptide is for example an antibody (immunoglobulin), the second antibody includes for example antibodies specifically binding to the immunoglobulin in a manner dependent on an animal from which the immunoglobulin is derived or on the class of the immunoglobulin. When the first polypeptide (primary antibody) is murine immunoglobulin (mouse-derived IgG1), for example, an anti-mouse IgG1 antibody can be used as the second polypeptide (secondary antibody). The second polypeptide is labeled or is to be labeled with a labeling substance. The labeling substance possibly used therefor is as described above.

The method for labeling the “polypeptide” with a labeling substance is appropriately selected from known methods appropriate for the labeling substance, for example the glutaraldehyde method, the periodate crosslinking method, the maleimide crosslinking method, the carbodiimide method, and the activated ester method in case of labeling with enzymes; and the chloramine T method and the lacto-peroxidase method [see Zoku Seikagaku Jikken Koza (Biochemistry Experimental Lesson), 2 “Tanpakushitsu no Kagaku (Protein Chemistry) (2)”, Tokyo Kagaku Dojin (1987)] in case of labeling with radioactive elements. When biotin is used as such labeling substance, for example, the N-hydroxysucccinimide ester derivative or hydrazide derivative of biotin (see Avidin-Biotin Chemistry: A Handbook, p. 57-63, PIERCE CHEMICAL COMPANY, issued in 1994) can be used.

Preferably, the “polypeptide” is preliminarily labeled with a labeling substance.

The polypeptide bound to C2GnT in a sample can be detected by detecting the labeling substance.

As the method for detecting the labeling substance, a known detection approach can be appropriately selected, depending on the type of the labeling substance. When one substance of a specific binding pair (for example, biotin) is used as a labeling substance, for example, an enzyme (for example, peroxidase) bound to the other substance specifically binding to the one substance (for example, streptoavidin) is added to form the specific binding pair. Subsequently adding a substrate [for example, hydrogen peroxide, o-phenylenediamine, 3,3′,5,5′-tetramethylbenzidine (TMB), and diaminobenzidine] of the enzyme [in the case that the enzyme is peroxidase] to the specific binding pair, the level of the color development in the product via the enzyme reaction is measured to detect the labeling substance.

When a radioactive element, a fluorescent dye or a chemiluminescent substance is used as such labeling substance, for example, a method for counting or measuring radioactivity, fluorescent intensity, fluorescent polarization, emission intensity or the like is listed.

C2GnT in a sample can be detected quantitatively or qualitatively (the detection of the presence or absence of C2GnT) by such methods. In other words, “the results of the detection of C2GnT” may be quantitative or qualitative results, satisfactorily.

When the qualitative measurement of C2GnT (the detection of the presence or absence of C2GnT) is intended, the presence or absence of a labeling substance is examined and the detected presence if any can be used as it is as a result of the detection of C2GnT.

When the quantitative detection of C2GnT is desired, further, the radioactive count, the intensity of color development, the fluorescent intensity, the emission intensity and the like can be used as they are as markers of C2GnT.

In accordance with the inventive method, the cancer of which the relationship with the results of the detection of C2GnT is to be examined and which is a subject of the examination and prediction of prognosis is not specifically limited, as long as the cancer is a disease recognized as a cancer in the field of the art. Preferably, however, the cancer includes “one or two or more cancers selected from the group consisting of prostate cancer, testicular tumor and bladder cancer”. The contents of the “prognosis of cancer” in accordance with the present invention are not specifically limited, but preferably include the “possibility of cancer metastasis or recurrence”. Particularly, the “possibility of cancer recurrence” is preferable.

The relationship between the “results of the detection of C2GnT” and the “prognosis of cancer” is satisfactorily established as follows.

When the “possibility of cancer metastasis or recurrence” is high, C2GnT in a sample significantly increases as described above. When C2GnT (the amount of C2GnT) in a sample is detected at a given level or more, therefore, the relationship of the amount of C2GnT with “a high possibility of cancer metastasis” or “a high possibility of cancer recurrence” in future can be established. When C2GnT (the amount of C2GnT) in a sample is detected at a given level or less, adversely, the relationship of the amount of C2GnT with “a low possibility of cancer metastasis” or “a low possibility of cancer recurrence” in future can be established.

When a section specimen of a living tissue is used, for example, the living tissue with C2GnT detected at 10% or more of the entire cancer cells therein under microscopic observation is determined as positive (+), while the living tissue with C2GnT detected otherwise is determined as negative (−). In the case that a biological sample is positive (+), the relationship of the sample with “a high possibility of cancer metastasis or recurrence” can be established.

Because the prognosis of cancer after resection of its cancer tissues can be predicted by the inventive method carried out before the resection of the cancer tissues, very useful information for the determination of a therapeutic course, such as the presence or absence of the need of the resection of cancer tissues, the possibility of radical cure of cancer singly with total resection of such tissues, and the need of selection of another therapy. This further leads to the avoidance of any unnecessary medical treatment and to the provision of such information at high accuracy to such patients, advantageously for these patients's benefit. Therefore, the inventive method is preferably practiced before the resection of cancer tissues. Particularly, the inventive method is preferably carried out before “total resection”.

<2> Inventive Kit

The inventive kit is a kit for detecting the prognosis of cancer, including at least the following element (A):

(A) a first polypeptide capable of binding to C2GnT.

Additionally, the inventive kit for detecting the prognosis of cancer preferably includes at least the following element (B):

(B) a second polypeptide capable of specifically binding to the first polypeptide (A), the second polypeptide being labeled or capable of being labeled with a labeling substance.

The description of the “first polypeptide capable of binding to C2GnT” is identical to the description of the “polypeptide capable of binding to C2GnT”. The description of the “second polypeptide capable of specifically binding to the first polypeptide” is identical to the above description about the “second polypeptide”. Additionally, the meanings of other terms for the inventive kit are also identical to those described above.

Thus, the “polypeptide” herein referred to is preferably an antibody or a polypeptide having its antigen-binding site, as described above.

The examination and prediction of the prognosis of cancer with the inventive kit can be carried out according to the inventive method (corresponding to the case of carrying out the detection of C2GnT with the “polypeptide capable of binding to C2GnT”).

The inventive kit is not specifically limited as long as the inventive kit contains at least the elements and further may include a reagent for detecting the labeling substance, and the like.

Other than these elements described above, the inventive kit may include a rinse solution, and a solution for terminating an enzymatic reaction. Additionally, the inventive kit may include a positive control (QC control) so as to keep the intra-assay level of batches at a given level.

These elements can be placed in separate containers and stored as a kit for use according to the inventive method.

The present invention is now described more specifically in the following Examples, but the present invention is not at all limited to these Examples.

The following samples, reagents and the like were used in the Examples.

Reagents, etc.:

The anti-C2GnT antibody (polyclonal antibody) described by Skrincosky D., Kain R., El-Battari A, et al., J Biol. Chem., 272, 22695-22702 (1997) was used.

An anti-sLe^{x} antibody (CSLEX-1) generated by a hybridoma (ATCC accession No. HB-8580) was used.

The anti-GnT-V antibody described in Int. J Cancer, 91(5), 631-637 (2001) was used.

Histofine Simple Stain MAX-PO (under trade name) manufactured by Nichirei Corporation was used as the peroxidase-labeled anti-mouse Ig antibody as a secondary antibody. AEC Substrate Kit manufactured by Nichirei Corporation was used, which included a substrate and a color-developing agent for peroxidase.

### EXAMPLE 1

Prostate Cancer:

In order to examine the relationship between the staining of prostate tissue with the anti-C2GnT antibody and the malignancy level or prognosis of prostate cancer, the following experiment was performed.

Prostate tissues were collected by biopsy from T1 patients and T2 patients (in total of 69 cases) under planning of radical prostatectomy. These patients did not undergo hormone therapy preoperatively or postoperatively. The collected prostate tissues were fixed with formalin and embedded in paraffin, to prepare a paraffin-embedded section. The section specimen was stained immunohistologically.

The immunohistological staining was done as follows: overnight reaction with the anti-C2GnT antibody as a first antibody at 4° C., subsequent one-hour reaction with the peroxidase-labeled anti-mouse Ig antibody as a second antibody at room temperature and color-developing reaction with a color-developing substrate.

After the staining, the resulting section specimen was observed with an optical microscope, to determine the specimen as positive (+) when 10% or more of the total cancer cells were stained; otherwise, the cancer cells were determined as negative (−)

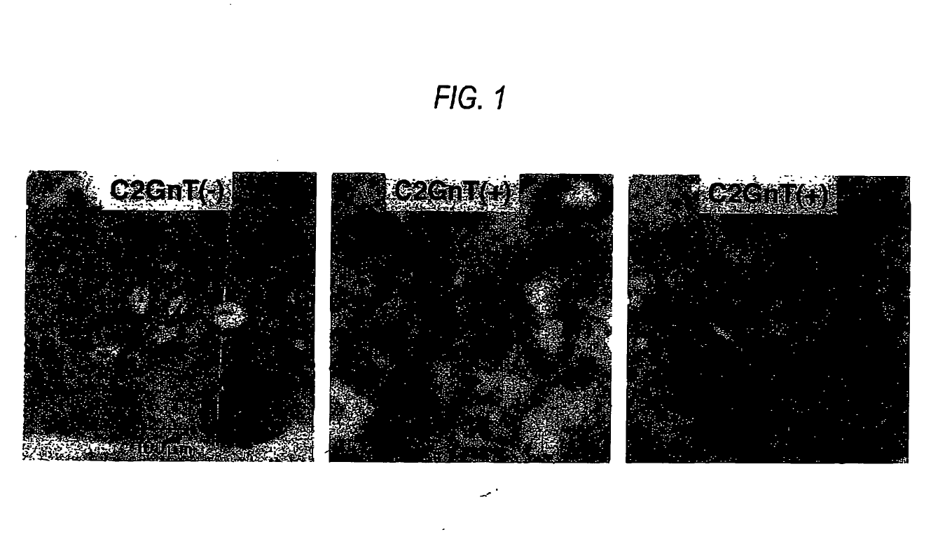

FIG. 1 shows one immunostaining example with the anti-C2GnT antibody. Because glycosyltransferase adding sugar chains locates intracellularly in the Golgi body, C2GnT is stained in particle forms in the vicinity of the nucleus. Generally, C2GnT is negative in a part with a small Gleason score, while C2GnT is stained as a distinct Golgi pattern in a part with a larger Gleason score.

(1) Relationship between Malignancy Level and C2GnT

The staining with the anti-C2GnT antibody (positive ratio) was compared with Gleason score. The results are shown in Table 1.

TABLE 1

| Gleason score | C2GnT negative | C2GnT positive |
|---|---|---|
| ≦6 | 12 | 6 (33%) |
| =7 | 8 | 18 (69%) |
| ≧8 | 2 | 23 (92%) |

(p = 0.0002)

As shown above, the C2GnT positive ratio was 6/18 (33%) in the group with Gleason scores of 6 or less, 18/26 (69%) in the group with the score of 7, or 23/25 (92%) in the group with Gleason scores of 8 or more. This indicates that a higher positive ratio of C2GnT significantly raises the Gleason score.

Using the anti-sLe^{x} antibody (CSLEX-1; prepared by the hybridoma of ATCC accession No. HB-8580) as a first antibody, the staining of sLe^{x} was examined. A similar result was obtained. However, the p-value for C2GnT was smaller than the p-value for sLe^{x}. Therefore, it is indicated that C2GnT more sensitively reflects the malignancy level.

(2) Relationship between Prognosis and C2GnT

(2-1) The staining with the anti-C2GnT antibody (positive ratio) was compared with pT (representing the stage of a pathological primary lesion; “pT2” represents that the primary lesion is confined to the inside of prostate, while “pT3” represents the state of the primary lesion breaking through the capsular membrane to infiltrate into the outside). Table 2 shows the results.

TABLE 2

| | C2GnT negative | C2GnT positive |
|---|---|---|
| pT2 | 21 | 23 (52%) |
| pT3 | 1 | 24 (96%) |

(p = 0.0001)

As shown above, the C2GnT positive ratio was 23/44 (52%) in the group diagnosed with “pT2” by the pT diagnosis of the biopsy specimen or 24/25 (96%) in the group diagnosed with “pT3”. Thus, it is indicated that a larger C2GnT positive ratio involves a higher pT stage.

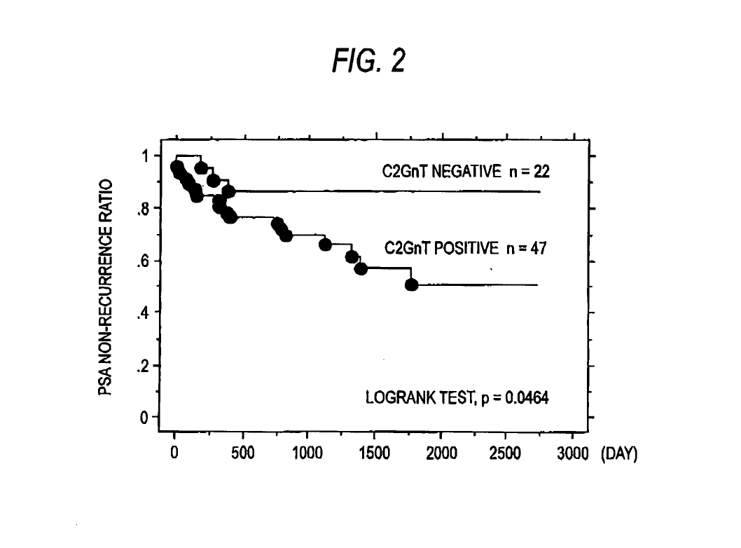

(2-2) The relationship between the staining with the anti-C2GnT antibody (positive ratio) and PSA failure was examined. FIG. 2 shows the relationship between the time period after prostatectomy and PSA failure (the PSA non-recurrence ratio).

FIG. 2 shows that the possibility of the occurrence of PSA failure in the C2GnT positive group after prostatectomy is higher than that of the C2GnT negative group (meaning that the PSA non-recurrence ratio in the C2GnT positive group is low). The result is statistically significant (p<0.0464 by the Logrank test).

Additionally, the frequency of PSA failure was 19/47 (40%) in the C2GnT positive group or 3/22 (13%) in the C2GnT negative group (the mean follow-up period of 38.3 months).

The results show the usefulness of C2GnT as a predictive factor of the pathological stage and a predictive factor of the occurrence of PSA failure after prostatectomy.

### EXAMPLE 2

Testicular Tumor:

In order to examine the relationship between the staining of testicular tumor tissue with the anti-C2GnT antibody and the malignancy level of testicular tumor or the like, the following experiment was performed.

133 cases with germ cell tumor out of 144 cases having undergone testis resection were subjects. The constitution is as follows.

Germ cell tumor: 133 cases
Seminoma: 68 cases
   Non-metastasis (stage I): 46 cases
   Metastasis (stage II or higher): 22 cases
Non-seminoma: 65 cases
   Non-metastasis (stage I): 28 cases
   Metastasis (stage II or higher): 37 cases

Paraffin-embedded section specimens were prepared from the collected testis tissues in the same manner as described above. Immunohistological staining with the anti-C2GnT antibody or the anti-GnT-V antibody as a first antibody was done in the same manner as described above.

After the staining, the specimens were observed with an optical microscope. When 10% or more of the total cancer cells were stained, the section was determined as positive (+); otherwise, the section was determined as negative (−).

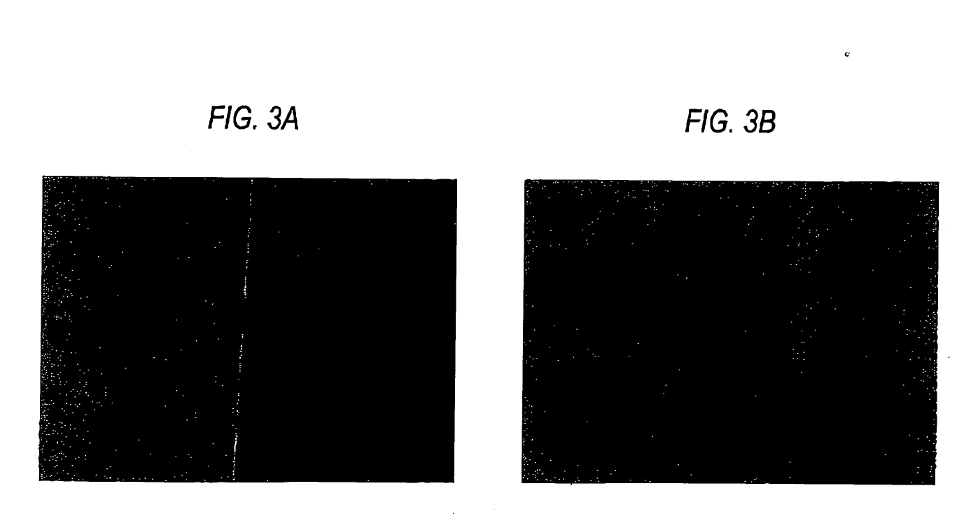

FIG. 3 shows one immunostaining example with the anti-C2GnT antibody. FIG. 3A shows the staining example of C2GnT-positive embryonal carcinoma, and FIG. 3B shows the staining of C2GnT-positive chorionic cancer.

(1) Relationship between Malignancy Level and C2GnT or GnT-V

The C2GnT positive ratio was 0/68 in the seminoma cases or 42/65 in the cases without seminoma. In other words, no expression of C2GnT in the seminoma case was observed.

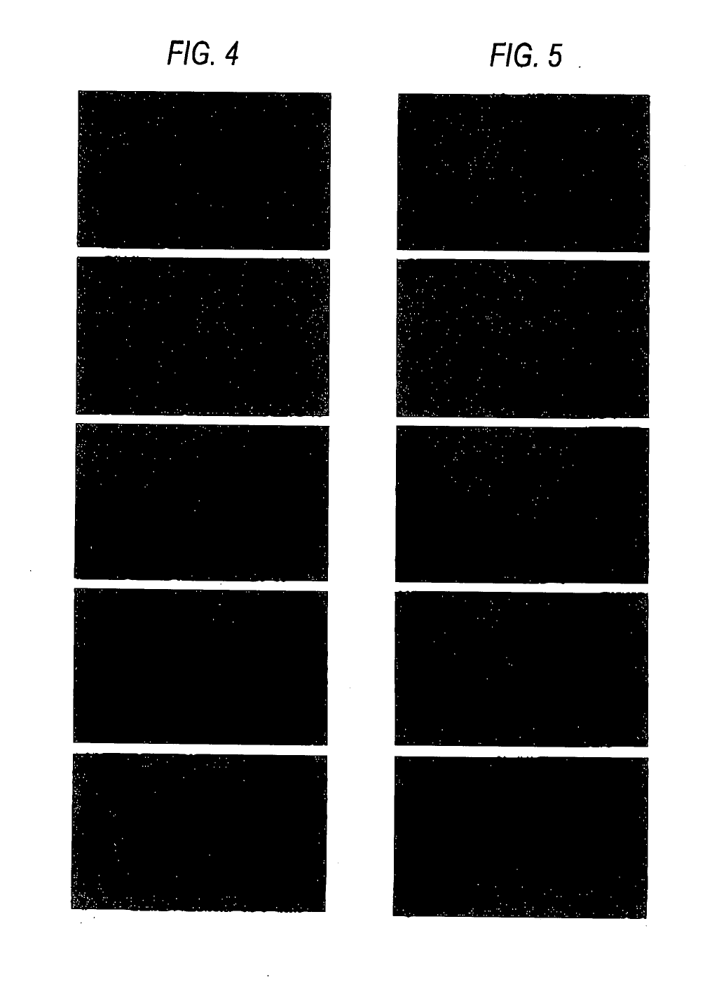

FIGS. 4 and 5 show one example of immunostaining of cancer tissues at various malignancy levels with the anti-C2GnT antibody (FIG. 4) or the anti-GnT-V antibody (FIG. 5). In both FIGS. 4 and 5, the malignancy level of tissues is in the ascending order from the upper panel to the lower panel.

Consequently, it is shown that a tissue with a higher GnT-V-stained level is at a lower malignancy level, while a tissue with a higher C2GnT-stained level is at a higher malignancy level. This indicates that C2GnT and GnT-V perform totally adversely to each other, from the standpoint of tissue malignancy level.

(2) Relationship between Metastasis and C2GnT

Concerning the presence or absence of the metastasis of testicular tumor tissue, individual patients in the C2GnT positive group and in the C2GnT negative group were examined. Then, the patients with or without metastasis were counted. The results are shown in Table 3.

TABLE 3

| | C2GnT negative | C2GnT positive | Total |
|---|---|---|---|
| Metastasis | 4 | 33 | 37 |
| Without metastasis | 19 | 9 | 28 |
| Total | 23 | 42 | 65 |

Table 3 shows a significantly greater number of metastatic cases in the C2GnT positive group.

(3) Relationship between Prognosis and C2GnT

The relationship between the staining with the anti-C2GnT antibody (positive ratio) and the non-recurrence ratio was examined. The relationship between the time period after testis resection and the non-recurrence ratio was as follows.

Even 35 months after the surgery, the non-recurrence ratio in the C2GnT negative group (n=19) was as high as 85%. In the C2GnT positive group (n=9), in contrast, gradual increase of recurrence was observed; 5 months after the surgery, the non-recurrence ratio was decreased down to 20%. The results indicate that the anti-C2GnT antibody positive cases had such a high recurrence ratio.

### EXAMPLE 3

Bladder Cancer:

In order to examine the relationship between the staining of bladder cancer tissue with the anti-C2GnT antibody and the malignancy level of bladder cancer or the like, the following experiment was performed.

Normal subjects and patients with bladder cancer (a total of 81 cases including the normal subjects) were examined. The constitution is as follows. Herein, “pT” expresses the depth of cancer invasion; pTa expresses the smallest depth; and a larger numerical figure attached to pT expresses a larger depth of cancer invasion.

Normal bladder: 15 cases

Bladder cancer: 66 cases
Papilloma: 2 cases
pTa: 23 cases
pT1: 20 cases
pT2 to pT4: 14 cases
Lymph node metastasis: 5 cases
Normal lymph node: 2 cases

The collected bladder tissues were prepared into paraffin-embedded section specimens in the same manner as described above. Immunohistological staining with the anti-C2GnT antibody or the anti-GnT-V antibody as a first antibody was done in the same manner as described above.

After the staining, the specimens were observed with an optical microscope. When 10% or more of the total cancer cells were stained, the section was determined as positive (+); otherwise, the section was determined as negative (−).

(1) Relationship between Malignancy Level and C2GnT or GnT-V

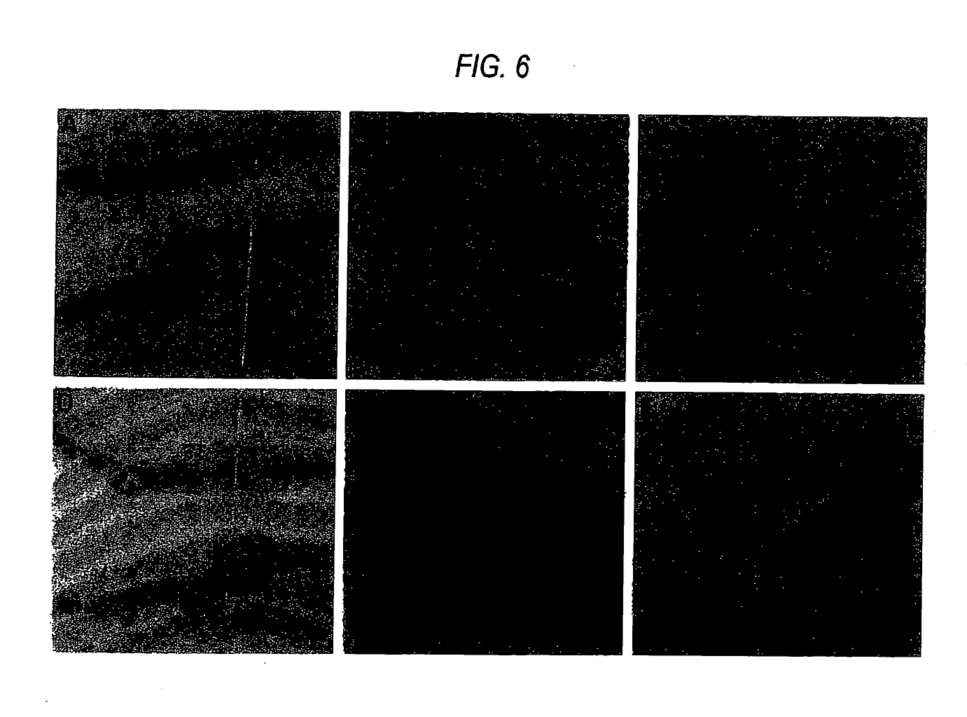

FIG. 6 shows each one example of immunostaining with the anti-C2GnT antibody and with the anti-GnT-V antibody. In FIG. 6, panels A through C show the immunostaining results with the anti-GnT-V antibody; and panels D through E show the immunostaining results with the anti-C2GnT antibody. The panels A and D represent tissues at the lowest malignancy level; the panels B and E, tissues at about moderate malignancy levels; and the panels C and F, tissues at the highest malignancy level.

Like testicular tumor, consequently, the results show that a tissue with a higher GnT-V-stained level is at a lower malignancy level, while a tissue with a higher C2GnT-stained level is at a higher malignancy level. This indicates that C2GnT and GnT-V perform totally adversely to each other, from the standpoint of tissue malignancy level.

Furthermore, the patients at various malignancy levels were counted in the GnT-V positive group and in the C2GnT positive group. The results are shown in Tables 4 and 5. In Table 5, “G1”, “G2” and “G3” express the levels of malignancy. A larger numerical figure means a higher level of malignancy.

TABLE 4

| | GnT-V | | C2GnT | |
|---|---|---|---|---|
| | positive | % | positive | % |
| Normal bladder | 0/15 | 0 | 0/15 | 0 |
| Papilloma | 2/2 | 100 | 0/2 | 0 |
| pTa | 19/23 | 82.6 | 2/23 | 8.7 |
| pT1 | 4/20 | 20.0 | 8/20 | 40.0 |
| pT2 to pT4 | 1/14 | 7.1 | 9/14 | 64.3 |
| Lymph node metastasis | 0/5 | 0 | 5/5 | 100.0 |
| Normal lymph node | 0/2 | 0 | 0/2 | 0.0 |

TABLE 5

| | GnT-V | | C2GnT | |
|---|---|---|---|---|
| | positive | % | positive | % |
| G1 | 10/12 | 83.3 | 0/12 | 0 |
| G2 | 13/29 | 44.8 | 12/29 | 41.4 |
| G3 | 1/21 | 4.7 | 12/21 | 57.1 |

These results indicate that the malignancy level (invasion level) in the GnT-V positive group is significantly low, while the malignancy level in the C2GnT positive group is significantly high.

### EXAMPLE 4

Preparation of the Inventive Kit:

The inventive kit of the following constitution was prepared.

1. Anti-C2GnT antibody (mouse): one bottle (first antibody)

2. Peroxidase-labeled anti-mouse Ig antibody: one bottle (second antibody)

3. TMB solution: one bottle (substrate)

While the present invention has been described in detail and with reference to specific embodiments thereof, it will be apparent to one of skill in the art that various changes and modifications can be made therein without departing from the spirit and scope thereof. All references cited herein are incorporated in their entirety.

This application is based on U.S. provisional patent application No. 60/455,585 filed on Mar. 19, 2003, the entire contents of which are incorporated hereinto by reference.

### INDUSTRIAL APPLICABILITY

The inventive method is very useful because the inventive method enables simple and rapid examination and prediction of the prognosis of cancer at high accuracy and low cost. Additionally, the inventive kit when used serves to carry out the inventive method in a far simpler and more rapid manner, so the inventive kit is very useful. At the time pre-treatment, the prognosis post-treatment can be predicted accurately in accordance with the present invention. The present invention provides extremely useful information for the determination of a therapeutic course for example for prostate cancer, such as the presence or absence of the need of for example prostatectomy, the possibility of radical cure of prostate cancer by prostatectomy alone, the need of selection of another therapeutic treatment, and the like. Owing to the present invention, further, any unnecessary medical treatment can be avoided and such information can be provided at high accuracy to patients to advantageously give benefits to these patients. By overall evaluation of the results from the practice of the inventive method in combination with parameters such as Gleason score and PSA value, the malignancy level of cancer, the progression thereof, the prognosis thereof, the presence or absence of lymph node metastasis, the presence or absence of recurrence can be predicted more accurately. Thus, the inventive method is extremely useful.

1. A method for detecting prognosis of cancer, which comprises at least a step of detecting core-2 β1,6-N-acetylglucosaminyltransferase in a sample collected from a biological organism to examine the relationship between the results of the detection and the prognosis of cancer in the biological organism.

2. The method according to claim 1, wherein the core-2 β1,6N-acetylglucosaminyltransferase is core-2 β1,6-N-acetylglucosaminyltransferase-I.

3. The method according to claim 1 or 2, wherein the biological organism is a human body.

4. The method according to any one of claims 1 to 3, wherein the sample is a living tissue.

5. The method according to any one of claims 1 to 4, wherein detecting of core-2 β1,6-acetylglucosaminyltransferase is carried out by using a polypeptide capable of binding to core-2 β1,6-N-acetylglucosaminyltransferase.

6. The method according to claim 5, wherein the polypeptide is an antibody or a polypeptide having its antigen-binding site.

7. The method according to any one of claims 1 to 6, wherein the cancer is one or at least two cancers selected from the group consisting of prostate cancer, testicular tumor and bladder cancer.

8. The method according to any one of claims 1 to 7, wherein the prognosis of cancer is possibility of cancer metastasis or recurrence.

9. The method according to any one of claims 1 to 8, which is carried out before resection of a cancer tissue.

10. The method according to claim 9, wherein the resection is total resection.

11. A kit for detecting prognosis of cancer, which comprises at least the following element (A):
(A) a first polypeptide capable of binding to core-2 β1,6-N-acetylglucosaminyltransferase.

12. The kit according to claim 11, which further comprises at least the following element (B):
(B) a second polypeptide capable of specifically binding to the first polypeptide described in (A), and being labeled or capable of being labeled with a labeling substance.

13. The kit according to claim 11 or 12, wherein the polypeptide is an antibody or a polypeptide having its antigen-binding site.
